(19) United States
(12) Patent Application Publication
(10) Pub. No.: US 2002/0001617 A1
(43) Pub. Date: Jan. 3, 2002

(54) Rapidly disintegrating tablet and process for the manufacture thereof

(76) Inventors: Chang-Hyun Lee, Kyungki-do (KR); Jong-Soo Woo, Kyungki-do (KR); Hee-Chul Chang, Kyungki-do (KR)

Correspondence Address: David A. Einhorn, Esq., Anderson Kill & Olick, P.C., 1251 Avenue the the Americas, New York, NY, 10020, US

(21) Appl. No.: 09/865,264

(22) Filed: May 25, 2001

(30) Foreign Application Priority Data
May 26, 2000 (KR) 2000-28667

(51) Int. Cl. A61K 9/20

(52) U.S. Cl. 424/465

(57) ABSTRACT

A tablet having an enhanced strength as well as a high disintegrating rate in the oral cavity is prepared by mixing a spray-dried particulate containing an active ingredient, a sublimable substance suitable for oral administration, a poly(ethylene glycol), and a pharmaceutically acceptable additive; tableting the mixture; and drying the resulting tablet to sublime the sublimable substance until the tablet becomes porous.

### FIELD OF THE INVENTION

The present invention relates to a rapidly disintegrating tablet for oral administration which has an enhanced strength as well as a high disintegrating rate in the oral cavity, and a process for the manufacture thereof.

### BACKGROUND OF THE INVENTION

Preparations for oral administration normally come in the form of tablet, granule, powder or solution. Since a solid preparation need be swallowed with some water, a liquid preparation is normally preferred by the elderly, infants or patients who have difficulty in swallowing. In spite of such advantage, a liquid preparation has shortcomings in that it is difficult to handle, especially in measuring an accurate dosage, and that it is not suitable for drugs which are unstable in a moist environment. Therefore, efforts have been made to develop a rapidly disintegrating tablet which easily disintegrates by the action of saliva.

There have been commercialized rapidly disintegrating tablets prepared by lyophilizing solutions containing various drugs (U.S. Pat. Nos. 5,631,023 and 5,976,577), e.g., Pepcid® RPD(famotidine preparation, Merck) and Zofrano zydis(ondansetron preparation, Glaxo wellcome), Claritin® RediTabs(loratadine preparation, Schering). However, these tablets have the disadvantage in that the productivity of the process for the preparation thereof is very low because the process involves the steps of injecting a drug solution into a pre-formed container, lyophilizing and coating the lyophilized product with an expensive material.

Instead of lyophilization, Yamanouch Pharmaceutical Co. Ltd. has disclosed in WO 99/47126 a rapidly disintegrating tablet prepared by using a water-soluble non-saccharide polymer as a binder together with an active ingredient; and humidifying the tablet. Further, WO 93/12769 discloses a rapidly disintegrating tablet prepared by filling a mold with a suspension containing an active ingredient together with agar and sugar; and drying the suspension to remove the solvent at 30° C. in a vacuum. However, these processes suffer from low productivity and uneven product quality.

Cima Labs has developed Orasolv technique which is disclosed in U.S. Pat. Nos. 5,173,878 and 6,024,981. Among the tablets prepared thereby, Zomig® Rapimelt(zolmitriptan preparation, Astrazeneca) has been commercialized. This tablet contains an effervescent substance but has the problems of incomplete disintegration in the oral cavity and the displeasing effect of the effervescent gas generated in the oral cavity.

U.S. Pat. No. 3,885,026 discloses porous tablets prepared by adding a volatilizable adjuvant, e.g., urethane, urea, ammonium carbonate or naphthalene, to other tablet components; tableting the resulting mixture; and heating the tablets to volatilize the adjuvant. However, a residual amount of the adjuvant in the tablet may generate a deleterious effect on the patient.

U.S. Pat. No. 4,134,943 discloses porous tablets prepared by adding a liquid having a freezing temperature in the range of −30 to 25° C. to other tablet components; cooling the mixture below the freezing temperature to solidify the liquid; tableting the cooled mixture; and then evaporating the liquid. However, this process suffers from low productivity.

### SUMMARY OF THE INVENTION

Accordingly, it is an object of the present invention to provide an improved process for preparing a rapidly disintegrating tablet which can be handled easily.

It is another object of the present invention to provide a rapidly disintegrating tablet prepared by said process.

In accordance with one aspect of the present invention, there is provided a process for preparing a rapidly disintegrating tablet which comprises the steps of: mixing a spray-dried particulate containing an active ingredient, a sublimable substance which is allowable for oral administration, a poly(ethylene glycol), and a pharmaceutically acceptable additive; tableting the mixture; and drying the resulting tablet to sublime the sublimable substance until the tablet becomes porous.

### BRIEF DESCRIPTION OF THE DRAWINGS

The above objects and features of the present invention will become apparent from the following description of preferred embodiments taken in conjunction with the accompanying drawings, in which:

FIGS. 1A to 1D show in vitro release profiles of the inventive tablet, the comparative tablet, and Zofran® zydis at pH 1.2, 4.0, 6.8 and water, respectively.

### DETAILED DESCRIPTION OF THE INVENTION

A composition which is used in preparation of the tablet of the present invention comprises a spray-dried particulate containing an active ingredient, a sublimable substance which is allowable for oral administration, a poly(ethylene glycol), and a pharmaceutically acceptable additive such as saccharide, surfactant, excipient and lubricant.

(1) Spray-dried Particulate Containing an Active Ingredient

The term “particulate” as used in the present invention means a substance comprised of particles of any shape.

The particulate used in the present invention may be obtained by dissolving an active ingredient, optionally together with a binder, an inorganic substance or a mixture thereof, in an appropriate solvent, e.g., water, ethanol or methanol, and drying the resulting solution using a conventional spray drying method.

The active ingredient which may be used in the tablet of the present invention include any pharmacologically active ingredients which can be orally administered, and preferred are those which dissolve rapidly in the oral cavity, the examples thereof being listed below:

\mathring{1} Antifebrile, analgesic or anti-inflammatory agents, e.g., aspirin, acetaminophen, indomethacin, sodium diclofenac, ketoprofen, isopropyl antipyrine, phenacetin, flurbiprofen and phenyl butazone;

\mathring{2} Anti-gastric ulcer agents, e.g., cimetidine, famotidine, ranitidine and nizatidine;

\mathring{3} Cardiovascular agents or vasodilants, e.g., nifedipine, almodipine, verapamil, captopril, diltiazem HCl, propranolol, oxprenolol, nitroglycerin and enalapril maleate;

\mathring{4} Antibiotics, e.g., cephalosporins such as ampicillin, amoxicillin and cephalexin; erythromycin; tetracycline; and quinolones;

\mathring{5} Antitussives or antiasthmatics, e.g., theophylline, aminophylline, codeine phosphate, methylephedrine HCl, dextromethorphan, noscapine, salbutamol, ambroxol, clenbuLerol and terbutaline;

\mathring{6} Antiemetics or stomach function-regulating agents; e.g., ondansetron, metoclopyramide, domperidone, trimebutine maleate, cisapride and levosulpiride;

\mathring{7} Impotence-treating, agents, e.g., agents that block the cleavage of nitrogen monoxide, including sildenafil, preferably a water soluble salt thereof; and

\mathring{8} Others which include a migrain-treating agent such as zolmitriptan and rizatriptan; a psychostimulant; an antibacterial agent; an antihistamines such as loratadine; antidiabetic; an allergy-treating agent; a contraceptive; a vitamin; an anticoagulant; a muscle-relaxing agent; a cerebral metabolism-improving agent; an antidiuretic; an anticonvulsant; and a Parkinson disease-treating agent such as selegiline.

The binder which may be used in the preparation of the spray-dried particulate gives the tablet the strength necessary for good handling and storage stability. Representative binders include polyvinylpyrrolidone, a copolymer of vinylpyrrolidone and vinylacetate, hydroxypropyl cellulose, hydroxypropyl methylcellulose, arabia gum, tragacanth gum, xanthan gum, sodium alginate, pectin, agar, water-dispersible starch and derivatives thereof, and a mixture thereof.

Representative inorganic substances include silicon dioxide, hydrotalcite, aluminum magnesium silicate, aluminum hydroxide, titanium dioxide, talc, aluminum silicate, magnesium aluminum metasilicate, bentonite and a mixture thereof.

The active ingredient may be combined with such a binder, an inorganic substance or a mixture thereof in a weight ratio ranging from 1:0.1 to 1:10, preferably 1:0.3 to 1:3. When the active ingredient particulate contains a binder, an inorganic substance or a mixture thereof, the active ingredient in the composition becomes more readily soluble and the taste of the drug can be blocked. Therefore, such a particulate is suitable for a drug having a poor solubility in water or bitter taste.

The amount of the spray-dried particulate used in preparing the inventive composition may be adjusted so that the content of the active ingredient is in the range of 0.5 to 80% by weight, preferably 1 to 70% by weight, based on the weight of the composition.

(2) Sublimable Substance

The sublimable substance which may be used in the present invention is a substance that causes no harmful effects when administered orally. The sublimable substance is tableted together with a spray-dried particulate containing an active ingredient, a poly(ethylene glycol), and pharmaceutically acceptable additives and then the resulting tablet is dried. During the drying process, the sublimable substance is sublimed to generate pores in the tablet. The porous tablet so obtained easily disintegrates in the oral cavity.

To accomplish such effect, the sublimable substance has to be sublimed at a temperature ranging from 40 to 60° C., preferably 40 to 50° C., more preferably 42 to 48° C., to prevent any property change of the saccharide. Further, since a residual amount of the substance may remain in the tablet after the drying process, it should not have a bad taste in addition to the requirement of being harmless. In the drying process, a reduced pressure may be employed in order to enhance the sublimation.

Representative sublimable substances which may be suitably used in the present invention include menthol; camphor; thymol; an organic acid such as adipic acid; and a lower fatty acid, e.g., arachidic acid, capric acid, myristic acid and palmitic acid, and a mixture thereof: and, among these, menthol is preferred.

The sublimable substance is used in an amount of 5 to 50% by weight, preferably 10 to 40% by weight, based on the weight of the composition.

(3) Poly(ethylene glycol)

The poly(ethylene glycol) which may be used in the present invention has a weight average molecular weight ranging from 1,000 to 20,000, preferably 1,500 to 10,000. The poly(ethylene glycol) enhances the dissolution of the drug and the abrasion resistance of the tablet. The poly(ethylene glycol) is used in an amount of 1 to 15% by weight, preferably 2 to 10% by weight, based on the weight of the composition.

(4) Saccharide

A saccharide having a sweet taste and good solubility in water may be used in the present invention. Representative saccharides include lactose, mannitol, sorbitol, xylitol, erythritol, glucose, sucrose, fructose, rebulose, maltodextrin, paratinose, and a mixture thereof. The saccharide may be used in an amount of 10 to 95% by weight, preferably 20 to 90% by weight, based on the weight of the composition.

(5) Surfactant

The surfactant may be used as a dissolution-supplementing agent in the composition. Representative surfactants include polyoxyethylene glycolated natural or hydrogenated vegetable oils such as Cremophor®(BASF); polyoxyethylene-sorbitan fatty acid ester such as Tween®(ICI); polyoxyethylene-polyoxypropylene block copolymer such as Poloxamer®(BASF); sorbitan fatty acid ester such as Span®(ICI); sodium lauryl sulfate; phospholipid and a mixture thereof. The surfactant may be used in an amount of 0.2 to 5% by weight, preferably 0.3 to 3.0% by weight, based on the composition.

(6) Others

In addition to the saccharide and the surfactant, the pharmaceutically acceptable additives which may be used in the present invention further include a disintegrator, e.g., cross-linked polyvinylpyrrolidone, sodium starch glycolate or calcium carboxymethyl cellulose; a lubricant, e.g., magnesium stearate, talc, silica, sodium stearyl fumarate or valine; a sweetening agent, e.g., aspartame, stevioside; an excipient, e.g., microcrystalline cellulose; and a mixture thereof. Each additive may be used in an amount of 0.1 to 20% by weight, preferably 0.2 to 10% by weight, based on the weight of the composition.

The tablet of the present invention is prepared by mixing a spray-dried particulate containing an active ingredient, a sublimable substance which is allowable for oral administration, an poly(ethylene glycol), and pharmaceutically acceptable additives; tableting the mixture; and drying the resulting tablet at a temperature ranging from 40 to 60° C., preferably 40 to 50° C., more preferably 42 to 48° C.

The following Examples are intended to further illustrate the present invention without limiting its scope.

### EXAMPLE 1

| Ingredients | Amount (mg/tablet) |
|---|---|
| Ondansetron | 8 |
| Menthol | 27 |
| Mannitol | 104.4 |
| Xylitol | 100 |
| Poly(ethylene glycol) 3000 | 5.5 |
| Poly(ethylene glycol) 6000 | 4.0 |
| Stevioside | 5.5 |
| Cross-linked polyvinylpyrrolidone | 4 |
| Magnesium Stearate | 1.2 |
| Silicon dioxide | 0.65 |

Ondansetron was dissolved in methanol and the solution was subjected to spray drying to obtain a particulate material. The particulate was mixed with the remaining ingredients and the resulting mixture was tableted. The resulting tablet was dried at 45° C. for 24 hours to sublime menthol until the content of residual menthol became 1 mg or less, to obtain a rapidly disintegrating tablet.

The fracture strength of the tablet was measured by applying a force (in g) against the tablet in the diametric direction using a loading plunger (diameter 1 cm) moving at a velocity of 0.5 mm/sec, and the force need to fracture the tablet (fracture strength) was observed to be approximately 220 g.

The disintegration time of the tablet in the oral cavity was determined by placing a tablet into a human mouth; and measuring the time period taken for complete disintegration of the tablet by saliva. This procedure was repeated 5 times using 5 separate individuals and a mean disintegration time was calculated from 3 data points omitting the longest and shortest time values. The resulting disintegration time was 25 seconds.

### EXAMPLE 2

| Ingredients | Amount (mg/tablet) |
|---|---|
| Ondansetron | 8 |
| Xanthan gum | 6 |
| Menthol | 29 |
| Mannitol | 104.4 |
| Polyethylene glycol 3000 | 9.5 |
| Stevioside | 5.5 |
| Cross-linked polyvinylpyrrolidone | 4 |
| Magnesium Stearate | 1.2 |
| Silicon dioxide | 0.65 |

Using the above ingredients, the procedure of Example 1 was repeated except that ondansetron and xanthan gum were used in the preparation of the particulate, to obtain a rapidly disintegrating tablet.

The fracture strength of the tablet was approximately 220 g and the disintegrating time of the tablet in the oral cavity was approximately 25 seconds.

### COMPARATIVE EXAMPLE

The procedure of Example 1 was repeated except that the ingredients were simply mixed without the step of preparing the particulate, to obtain a porous tablet (comparative tablet).

The fracture strength of the porous tablet was approximately 230 g and the disintegrating time of the porous tablet in the oral cavity was approximately 25 seconds.

### TEST EXAMPLE

Dissolution Test

A dissolution test was conducted for the tablets obtained in Example 1 and Comparative Example as well as Zofran® zydis (Glaxo wellcome) as a control, in accordance with the dissolution test method described in Korean Pharmacopoeia by the Korea Food and Drug Administration (KFDA) under the conditions listed below:

Test apparatus: ERWEKA DT80 (Erweka, Germany)

Analytical method: liquid chromatography

column: Inertsil ODS-2(4.6×150 mm; GL Science, Japan)

mobile phase: Acetonitrile: 0.02M KH₂PO₄=30:70

flow rate: 1.0 ml/min.

detector: UV 278 nm

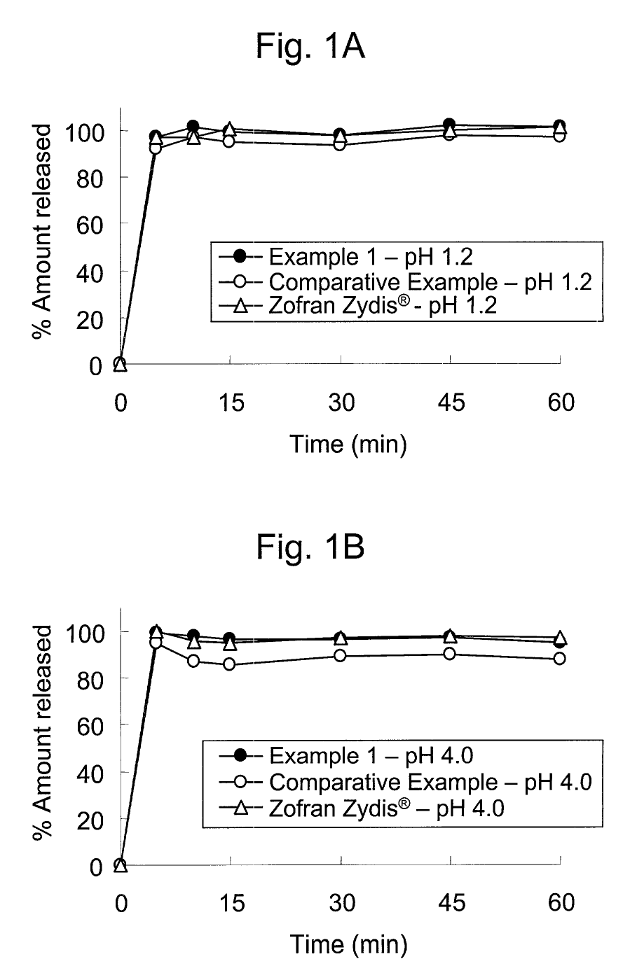

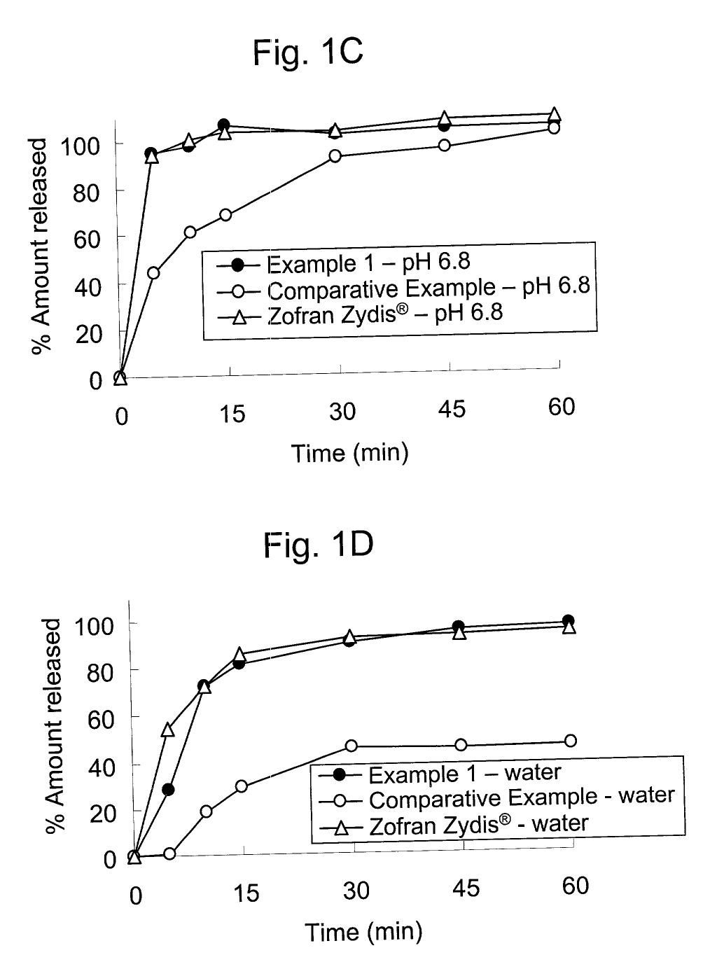

FIGS. 1A to 1D show in vitro release profiles of the inventive tablet, the comparative tablet, and Zofran® zydis at pH 1.2, 4.0, 6.8, and water, respectively.

As can be seen from FIGS. 1A to 1D, the inventive tablet shows dissolution rate comparable to the Zofran® zydis control. In contrast, the comparative tablet exhibits an inferior dissolution rate.

While the invention has been described with respect to the above specific embodiments, it should be recognized that various modifications and changes may be made to the invention by those skilled in the art which also fall within the scope of the invention as defined by the appended claims.

What is claimed is:

1. A process for preparing a rapidly disintegrating tablet which comprises: mixing a spray-dried particulate containing an active ingredient, a sublimable substance suitable for oral administration, a poly(ethylene glycol), and a pharmaceutically acceptable additive; tableting the mixture; and drying the resulting tablet to sublime the sublimable substance until the tablet becomes porous.

2. The process of claim 1, wherein the spray-dried particulate is prepared by dissolving the active ingredient in a solvent and spray drying the resulting solution.

3. The process of the claim 1, wherein the spray-dried particulate contains an active ingredient selected from the group consisting of: an analgesic selected from the group consisting of aspirin, acetaminophen, indomethacin, sodium diclofenac, ketoprofen, isopropyl antipyrine, phenacetin, flurbiprofen and phenyl butazone; an anti-gastric ulcer agent selected from the group consisting of cimetidine, famotidine, ranitidine and nizatidine; a cardiovascular agent selected from the group consisting of nifedipine, almodipine, verapamil, captopril, diltiazem HCl, propranolol, oxprenolol, nitroglycerin and enalapril maleate; an antibiotic selected from the group consisting of ampicillin, amoxicillin, cephalexin, erythromycin, tetracycline, and quinolone; an antiasthmatic selected from the group consisting of theophylline, aminophylline, codeine phosphate, methylephedrine HCl, dextromethorphan, noscapine, salbutamol, ambroxol, clenbuterol and terbutaline; an antiemetic agent selected from the group consisting of ondansetron, metoclopyramide, domperidone, trimebutine maleate; a stomach function-regulating agent selected from the group consisting of cisapride and levosulpiride; an impotence-treating agent; a migrain-treating agent selected from the group consisting of zolmitriptan and rizatriptan; a psychostimulant; an antibacterial agent; an antihistamines; an antidiabetic; an allergy-treating agent; a contraceptive; a vitamin; an anticoagulant; a muscle-relaxing agent; a cerebral metabolism-improving agent; an antidiuretic; an anticonvulsant; and a Parkinson disease-treating agent.

4. The process of claim 3, wherein the spray-dried particulate further contains a binder, an inorganic substance or a mixture thereof.

5. The process of claim 4, wherein the binder is selected from the group consisting of polyvinylpyrrolidone, a copolymer of vinylpyrrolidone and vinylacetate, hydroxypropyl cellulose, hydroxypropyl methylcellulose, arabia gum, tragacanth gum, xanthan gum, sodium alginate, pectin, agar, water-dispersible starch and its derivatives, and a mixture thereof.

6. The process of claim 4, wherein the inorganic substance is selected from the group consisting of silicon dioxide, hydrotalcite, aluminum magnesium silicate, aluminum hydroxide, titanium dioxide, talc, aluminum silicate, magnesium aluminum metasilicate, bentonite and a mixture thereof.

7. The process of claim 4, wherein the active ingredient and, the binder, the inorganic substance or the mixture thereof are used in a weight ratio ranging from 1:0.1 to 1:10.

8. The process of claim 1, wherein the sublimable substance is selected from the group consisting of menthol, camphor, thymol, an organic acid, a lower fatty acid and a mixture thereof.

9. The process of claim 1, wherein the poly(ethylene glycol) has a weight average molecular weight ranging from 1,000 to 20,000.

10. The process of claim 1, wherein the mixture comprises 0.5 to 80% by weight of the active ingredient in the particulate form, 5 to 50 by weight of the sublimable substance and, 1 to 15 by weight of the poly(ethylene glycol), based on the weight of the mixture.

11. A rapidly disintegrating tablet prepared by the process of any one of claims 1 to 10.
